(19) United States
(12) Patent Application Publication
(10) Pub. No.: US 2023/0346012 A1
(43) Pub. Date: Nov. 2, 2023

(54) MINITOX 2 MOUTHPIECE WITH VENTILATED AIRFLOW FOR A SMOKING ARTICLE

(71) Applicant: Eldar Bakhram SARIYEV, Baku (AZ)

(72) Inventors: Eldar Bakhram Sariyev, Baku (AZ); Leyla Eldar Sari, Baku (AZ)

(21) Appl. No.: 17/926,363

(22) PCT Filed: May 20, 2020
(86) PCT No.: PCT/AZ2020/000004

§ 371 Date: Nov. 18, 2022

(51) Int. Cl. A24F 7/04; A24F 1/30; A24F 7/02; B01D 53/04; B01J 20/08; B01J 20/10; B01J 20/16; B01J 20/28; A61L 9/014

(52) U.S. Cl. CPC A24F 7/04; A24F 1/30; A24F 7/02; B01D 53/0415; B01D 53/0446; B01J 20/08; B01J 20/103; B01J 20/165; B01J 20/28016; A61L 9/014; B01D 2253/104; B01D 2253/106; B01D 2253/102; B01D 2253/20; B01D 2257/80; B01D 2257/90; B01D 2257/93; B01D 2259/4541; A61L 2209/22

(57) ABSTRACT

A “MINITOX FAN” mouthpiece with a ventilated air flow for a smoking article comprises a housing, a smoking pipe, said housing being configured from two hollow conical detachable parts for connection to one another and to cylindrical appendices at opposite ends for connection to the smoking pipe and to a hose, respectively; a cylindrical cartridge with a cap, which is disposed inside the housing and has a closed bottom with a lip and through slots across the entire surface of the housing; an insert in the form of a hollow cylinder of smaller diameter for placement inside the cartridge, said insert having a closed bottom, a tightly sealing cap and through slots on the cylindrical surface, at each end of which are appendices, having a diameter that is not greater than the diameter of granules of aluminum oxide and silica gel, for suspended placement of the insert inside the cartridge, wherein the cartridge comprises segments containing a moisture-retaining sorbent and an odor-absorbing sorbent in the form of particles or granules, and the insert contains a detoxifying sorbent; and a fan in the form of a wheel with a shaft.

### FIELD OF THE INVENTION

The invention constitutes a part of the mouthpiece of a smoking article, in particular a mouthpiece with ventilation for mixing the main stream of smoke generated by using smoking articles with the surrounding air. The invention can be applied to smoking articles such as a hookah.

### BACKGROUND OF INVENTION

Hookah cannot be classified as a safe hobby, and in some cases the harm caused by hookah outweighs that of cigarettes. In addition to poisoning by the byproducts of combustion when smoking a hookah, there is a possibility of being infected with pathogenic microorganisms. This is especially true in the absence of a replaceable mouthpiece.

There are USA-made filter mouthpieces for hookahs that presumably reduce the level of nicotine in inhaled smoke, as well as filter mouthpieces reducing the rust and debris that may be present in old hoses or non-washable hoses [https://smokehookah.livejournal.com/9944.html].

There are also disposable hookah mouthpieces, which are in the form of a plastic attachment for a hookah pipe. This attachment can be easily and effortlessly removed and attached. They are external and internal. External disposable mouthpieces differ in that they must be put on top of the hose, while the inner ones are attached to the hookah hose from inside. [http://hookah4you.ru/mund-dlya-kalyana.html]

In the publication entitled “Mouthpiece of a Smoking Article, Including Two Materials in the Form of Particles” [RU 141067 U1, A24D 1/04 (2006.01), publ. 27 May 2014], a mouthpiece is proposed containing a segment including an absorbent material in the form of particles and cellulose material in the form beads or granules, evenly distributed among the particles of the absorbent material, which is different from the color of the specified absorbent material. It should be mentioned that the specified adsorbent material contains activated carbon, and the cellulosic material contains aluminum oxide. According to the application, the mentioned segment contains a fibrous filter material, and the aforementioned absorbent and cellulosic materials are dispersed within the fibrous filter material. The mouthpieces of this useful invention are preferably used in filter cigarettes and other smoking articles in which tobacco is burned producing smoke. The disadvantage of the well-known design lies in the fact that it is multicomponent, difficult to manufacture, and has low protection effectiveness.

The mouthpiece for the smoking item “MINITOX” [AZ U 2018 0023, A24D 3/06, Bulletin No. 10. Dec. 30, 2019] contains segments including the following: a detoxification sorbent in the form of particles or granules, a water-retaining sorbent in granular form (the mentioned water-retaining sorbent is located in the segment in the lower part of the mouthpiece from the side of the smoking article), a detoxification sorbent in the upper part of the mouthpiece from the side of the smoker's mouth, and the segments are separated from each other and from the lower and upper parts by a mesh material. The mouthpiece as a detoxification sorbent contains the coconut sorbent CARBOTECH DGK 8X30/60 and a harmala at a mass ratio of 1: 0.1-0.5 respectively. Additionally, as a water-retaining sorbent, it contains silica gel of the MSMK or ASMK brands or particles of aluminum oxide, or a mixture of silica gel.

The invention “A smoking article with a ventilated mouthpiece containing the first and second airflow streams” [RU 2 620 946, C2, A24D 3/04, May 30, 2017 Bull. No. 16] refers to a smoking article which is comprised of a mouthpiece surrounded by a first wrapper and a second wrapper, in which the wrappers are airtight and made to allow rotation between the wrappers along the longitudinal axis of the smoking article. Thus, the mouthpiece contains the first stream of airflow through the first or the second wrapper, and the second airflow stream through the first or second wrapper. In a configuration that has weak ventilation, the airflow in the mouthpiece through the first airflow stream is limited, and the airflow in the mouthpiece through the second airflow path is basically unlimited. However, in highly ventilated configurations, the airflow in the mouthpiece through the first and second airflow stream is basically unrestricted, while a minimum level of ventilation is provided by the airflow in the mouthpiece via one or both of the first airflow streams generated during the rotation of the first and second wrappers relative to each other between the weakly ventilated and highly ventilated configurations. The first wrapper and the second wrapper are one relative to the other between the low-ventilated configuration and the strong-ventilated configuration. The technical result consists of ensuring a constant level of ventilation in the position of the weakest ventilation. However, the invention is ineffective, as quite often in the course of smoking the wrappers are shifted to the position of closed holes and the ventilation system malfunctions. Moreover, the invention is intended for such smoking articles as cigarettes.

The hookah smoke has a high level of humidity. Therefore, in addition to detoxification, it creates the opposite effect of inhalation with toxic combustion products.

The aim of this technical solution is to reduce the toxicity of the smoking article while maintaining the organoleptic properties of the smoking mixture.

### SUMMARY OF THE INVENTION

The present invention proposes a mouthpiece for a smoking article with ventilated airflow “MINITOX FAN”, comprised of a frame, a smoking pipe (the frame is made of two hollow cone-shaped detachable parts; one in the lower part from the side of the smoking article with an internal thread, and one in the upper part from the side of the smoking pipe, made with a shoulder with an external thread, with a groove on the inner surface, and a cylindrical outgrowth on opposite sides for connection with a smoking pipe and a hose of a smoking article), a cylindrical cartridge located inside the frame—made with a blind bottom with a shoulder and a lid with perforating transverse slits along the entire surface—a receptacle made in the form of a hollow cylinder of a smaller diameter for placement inside the cartridge with a dead bottom and an airtight lid with perforating transverse slits on the cylindrical surface, both sides of which have outgrowths with a diameter of no more than the diameter of aluminum oxide and silica gel granules for its weighed placement in the cartridge (the cartridge contains segments including moisture-retaining and odor-absorbing sorbents, enclosed on both sides by a mesh partition, and the receptacle contains a sorbent for detoxification in the form of particles or granules), and a fan located in the groove of the upper part of the frame, made in the form of a wheel with a shaft, with three blades and a plug for fixing the fan blades.

The material for the frame, cartridge and receptacle is selected to be acrylonitrile, a transparent copolymer, with styrene butadiene ABS (Acrylonitrile-Butadiene-Styrene EC number 920-401-2, CAS number 9003-56-9), the fan is high-impact polystyrene. As a sorbent for detoxification, the mouthpiece contains coconut sorbent CARBOTECH DGK 8X30/60 and harmala at a mass ratio of 1:0.1 respectively; as a water-retaining sorbent it contains silica gel of the MSMK or ASMK brands, or particles of aluminum oxide, or a mixture of silica gel and particles of aluminum oxide at mass ratio 1:1, natural aluminosilicates are the agents that absorb odor sorbent. Additionally, the cartridge contains some segments with water-retaining and odor-absorbing sorbents, while the cartridge contains a sorbent for detoxification. The characteristics of the materials are described in the certificate (See Appendix).

The novelty of the invention lies in the design of the mouthpiece with a removable cartridge with sorbents and a fan, which allows the use of the smoking article with maximum safety and efficiency.

The inventive level is confirmed by a new technical result, in particular, the airflow generated by the fan during puffing cools the hookah aerosol and separates the vapor phase containing resinous carcinogenic compounds from the inhaled smoke, which provides additional protection for the body.

The essence of the invention lies in the adsorption of toxic gases and isotopes of heavy metals on the surface of the adsorbents, the separation of aerosol vapors with carcinogenic substances released from tobacco and tobacco products, as well as the simultaneous therapeutic effect (sedative, antispasmodic) provided by the harmala and enhancement of the organoleptic properties of smoking mixtures. Harmala (Péganum hármala) is a perennial, herbaceous plant of the Peganaceae family (known in standard technical documents as Zygophyllaceae parifolia family) growing as a weed in the southern regions of the European parts of the CIS and Western Siberia, in all of the Central Asian Republics and Southern Kazakhstan [P. Revvel, Ch. Revvel, Our Environment, Volume IV, publisher: Mir, pp.56-59] Harmala is utilized as a raw material in medicine. All parts of the plant contain alkaloids, derivatives of quinazoline and indole: harmine, harmanine, harmolol, peganin. In the budding phase, the alkaloid L-peganin predominates. The fruits contain 14-25% fatty oils. Raw materials are used to obtain a preparation of deoxypeganine hydrochloride, which has an anticholinesterase effect. The drug is used for lesions of the nervous system [Zh. A. Demirov et al. Plants and nature, Baku, Ganjlik, 1992, p. 105]. In Asian medicine, seeds are used as a narcotic, antispastic, hypnotic, for heart pain [F. Lemberani, Folk Medicine, Publishing House Logman, 2000, p. 76].

The silica gel that is utilized is of technical grade MSMK or ASMK GOST 3956-76, which has maximum photoabsorption and water retention.

For detoxification, the coconut sorbent CARBOTECH DGK 8X30/60 is used, which is activated carbon derived from the coconut shell.

### BRIEF DESCRIPTION OF THE DRAWINGS

The invention is illustrated in the following drawings.

FIG. 1 overview of the mouthpiece for the smoking article “MINITOX 2”

FIG. 2 the lower part of the mouthpiece frame.

FIG. 3 upper part of the mouthpiece frame.

FIG. 4 smoking pipe

FIG. 5 cartridge.

FIG. 6 receptacle.

FIG. 7 fan.

### DETAILED DESCRIPTION OF EMBODIMENTS

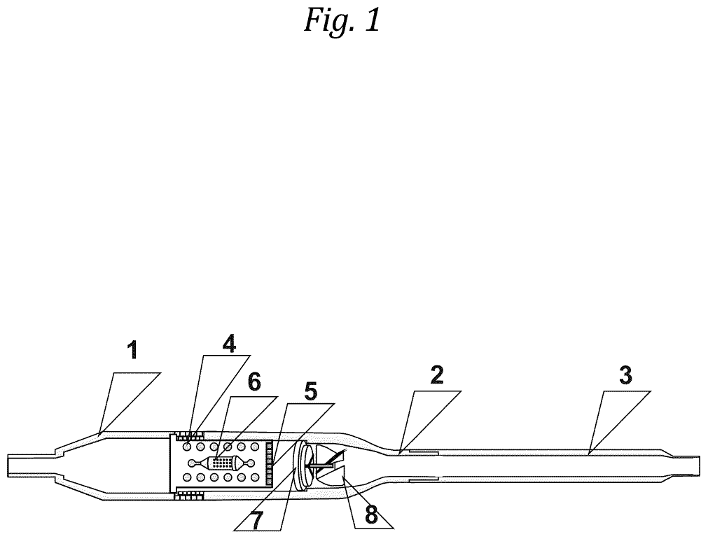

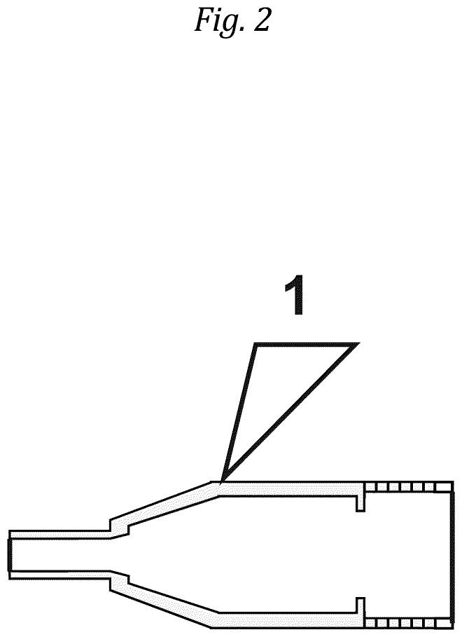

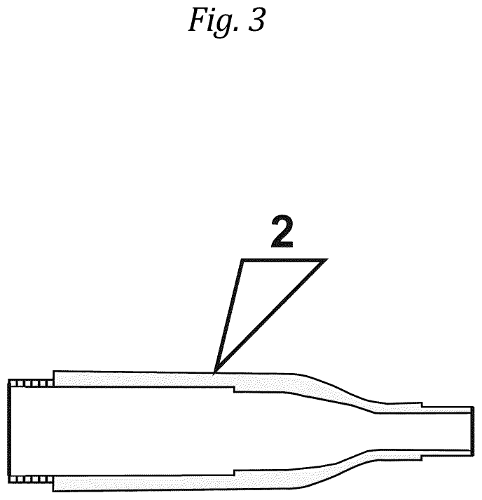

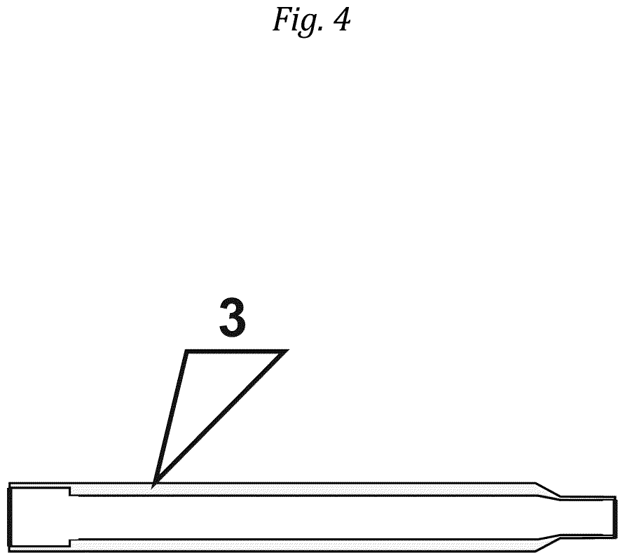

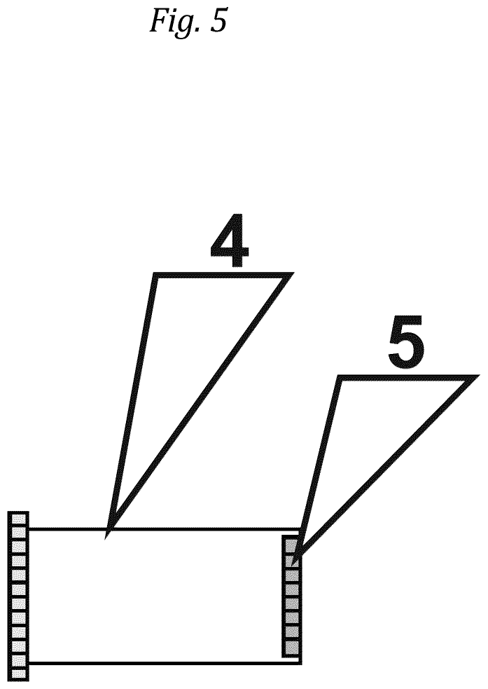

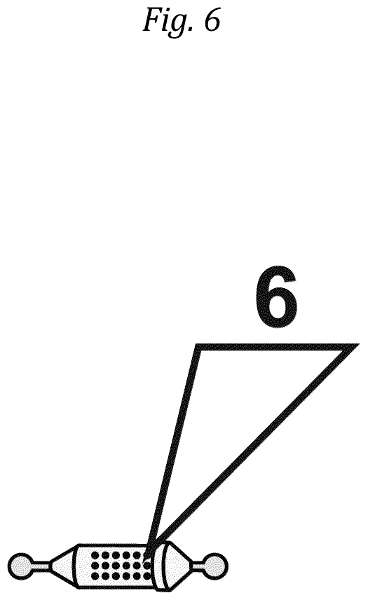

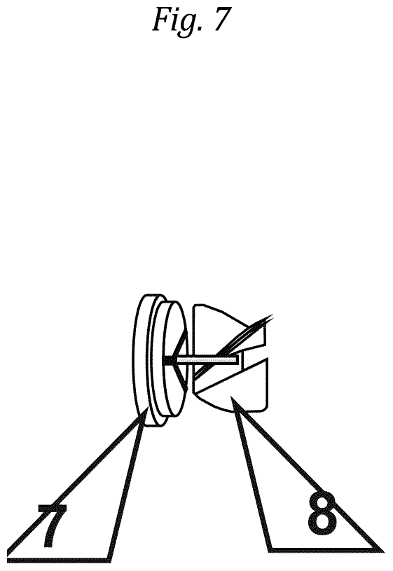

Smoking Article Mouthpiece with Vented Airflow “MINITOX FAN” (FIG. 1) contains a hollow cylindrical frame made of two hollow cone-shaped detachable parts: the lower part 1 (FIG. 2) from the side of the smoking article with an internal thread; and the upper part 2 (FIG. 3) from the side of the smoking pipe with a shoulder with an external thread, for their alignment with each other and with a cylindrical outgrowth on opposite sides for connection with the smoking pipe 3 (FIG. 4) and the hose of the smoking article; a cylindrical cartridge 4 (FIG. 5), made with a dead bottom with a shoulder, with perforating transverse slits along the entire surface, placed inside the frame, complete with a removable lid 5 with identical slits; receptacle 6 (FIG. 6), made in the form of a hollow cylinder of a smaller diameter for placement inside the cartridge with perforating transverse slits, with a dead bottom on one side and with an airtight lid on the other, on both sides of which there are appendages with a diameter of no more than the diameter of the granules of aluminum oxide and silica gel for its weighed placement in the cartridge; and a fan (FIG. 7), made in the form of a wheel 7 with a shaft, which includes three blades 8 and a plug (not shown in the drawing) for fixing the fan blades, while the fan is located in the groove of the upper part of the frame. The cartridge 4 contains segments enclosed on both sides by a mesh partition made of a synthetic mesh material (not shown in the drawing), including moisture-retaining and odor-absorbing sorbents in the form of particles or granules, and the cartridge contains a sorbent for detoxification in the form of granules.

The mouthpiece is used as follows:

The bottom of the cartridge is covered with a mesh partition. A receptacle with a mixture of CARBOTECH DGK 8X30/60 coconut sorbent and harmala at a mass ratio of 1:0.1 is lowered into it, thereafter the cartridge is filled with the sorbents of silica gel or particles of aluminum oxide, or a mixture of silica gel and particles of aluminum oxide at a mass ratio of 1:1, natural aluminosilicates, close the cartridge tightly with a mesh baffle plate and a lid. A fan is installed in the groove of the upper part of the frame, then the cartridge is installed up to the flange of the bottom to the flange of the upper part of the frame and the lower part of the frame is then screwed in its place. On one of the sides, the frame is connected with a hose to a smoking article, while on the other it is connected to a smoking pipe. The fan starts spinning during inhaling.

When inhaled, hot vapors of aerosol water are absorbed by silica gel and aluminum oxide, while toxic substances, such as tobacco combustion products, are retained by the sorbent for detoxification. The fan cools and separates the vapor phase containing tarry carcinogens from the inhaled smoke.

What is claimed is:

1. A mouthpiece for a smoking article with ventilated airflow, containing a frame, a smoking pipe (the frame is made of two hollow cone-shaped detachable parts: one in the lower part from the side of the smoking article with an internal thread, and one in the upper part from the side of the smoking pipe, made with a shoulder with an external thread, with a groove on the inner surface, and with a cylindrical outgrowth on opposite sides for connection with a smoking pipe and a hose of a smoking article), a cylindrical cartridge located inside the frame, made with a blind bottom with a shoulder and a lid with perforating transverse slits along the entire surface, a receptacle made in the form of a hollow cylinder of a smaller diameter for placement inside the cartridge with a dead bottom and an airtight lid with perforating transverse slits on the cylindrical surface, both sides of which have outgrowths with a diameter of no more than the diameter of aluminum oxide and silica gel granules for its weighed placement in the cartridge (the cartridge contains segments including moisture-retaining and odor-absorbing sorbents, enclosed on both sides by a mesh partition, and the receptacle contains a sorbent for detoxification in the form of particles or granules), and a fan located in the groove of the upper part of the frame, made in the form of a wheel with a shaft, with three blades and a plug for fixing the fan blades.

2. The mouthpiece for a smoking article according to article 1, in which the material of the frame, cartridge, and receptacle are selected to be of a transparent copolymer of acrylonitrile with styrene-butadiene ABS.

3. The mouthpiece for a smoking article according to article 1, where the fan is to be made from impact resistant polystyrene.

4. The mouthpiece for a smoking article according to article 1, which contains a mixture of coconut sorbent CARBOTECH DGK 8X30/60, and harmala at a mass ratio of 1:0.1 respectively as a sorbent for the purpose of detoxification.

5. The mouthpiece for a smoking article according to article 1, which contains silica gel of the MSMK or ASMK brands acting as a water-retaining sorbent.

6. The mouthpiece for a smoking article according to article 1, which contains particles of aluminum oxide as a water-retaining sorbent.

7. The mouthpiece for a smoking article according to article 1, which contains a mixture of silica gel and aluminum oxide particles at a mass ratio of 1:1, used as a water-retaining sorbent.

8. The mouthpiece for a smoking article according to article 1, which contains natural aluminosilicates as odor absorber.

9. The mouthpiece for a smoking article according to article 1, which has a cartridge equipped with a mesh baffle made of synthetic mesh material on both sides.
